SEQUENCE LISTING (6 sequences)

SEQ ID NO: 1 - DNA, 171 bp, Artificial Sequence
  CDS (1)..(168)
  Description of Artificial Sequence Synthetic nucleotide sequence encoding a fusion protein
atgagctccaattcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatag

SEQ ID NO: 2 - protein, 56 aa, Artificial Sequence
  Description of Artificial Sequence Synthetic fusion protein sequence
MSSNSLAVVLQRRDWENPGVTQLNRLAAHPPFASWRNSEEARTDRPSQQLRSLNGE

SEQ ID NO: 3 - DNA, 27 bp, Artificial Sequence
  Description of Artificial Sequence Synthetic primer
ccgaagcttatgttccaggcggccgag

SEQ ID NO: 4 - DNA, 27 bp, Artificial Sequence
  Description of Artificial Sequence Synthetic primer
ataggatcctaacgtcagacgctggcc

SEQ ID NO: 5 - DNA, 31 bp, Artificial Sequence
  Description of Artificial Sequence Synthetic primer
ataggatccatgagctccaattcactggccg

SEQ ID NO: 6 - DNA, 38 bp, Artificial Sequence
  Description of Artificial Sequence Synthetic primer
ataagaatgcggccgcctattcgccattcaggctgcgc

(19) United States
(12) Patent Application Publication
(10) Pub. No.: US 2004/0137480 A1
(43) Pub. Date: Jul. 15, 2004

(54) Monitoring intracellular proteins

(76) Inventor: Richard M. Eglen, Los Altos, CA (US)

Correspondence Address: PETERS VERNY JONES & SCHMITT, L.L.P., 425 SHERMAN AVENUE, SUITE 230, PALO ALTO, CA, 94306, US

(21) Appl. No.: 10/702,232

(22) Filed: Nov. 6, 2003

Related U.S. Application Data
(63) Continuation of application No. 10/229,747, filed on Aug. 27, 2002.
(60) Provisional application No. 60/353,086, filed on Jan. 30, 2002.
(60) Provisional application No. 60/316,428, filed on Aug. 30, 2001.

(30) Foreign Application Priority Data
Aug. 27, 2002 (WO) PCT/US02/27497

(51) Int. Cl. C12Q 1/68

(52) U.S. Cl. 435/6

(57) ABSTRACT

Methods and compositions are provided for analyzing intracellular events employing fusion proteins comprising the small β-galactosidase fragment fused to a protein of interest or fragment thereof. By expressing the fusion protein in a cellular host, one can determine the effect of various agents on the activity of the fusion protein, adding the large β-galactosidase fragment to the fusion protein, either intracellularly or in a lysate and determining the activity of the formed β-galactosidase using a substrate that forms a detectable product. In this manner, degradation, complex formation, RNAi inhibition or other situation that affects β-galactosidase activity can be determined.

### REFERENCE TO RELATED APPLICATIONS

This application is a continuation-in-part of provisional application 60/316,428, filed Aug. 30, 2001 and 60/353,086, filed Jan. 30, 2002, and a continuation of full application Ser. No. 10/229,747 filed Aug. 27, 2002 and PCT application PCT/US02/27497 filed Aug. 27, 2002 the contents of each of which is incorporated herein by reference.

### BACKGROUND OF THE INVENTION

1. Field of the Invention

The field of this invention is proteomics.

2. Background Information

The elucidation of the human genome and that of other species has greatly accelerated with the interest in proteomics, that is, the study of naturally occurring proteins and their intra- and extracellular interactions and activities. The ability to determine the status of a protein in a cell has far ranging opportunities in understanding the intracellular pathways, the intracellular movement of proteins into different compartments, the regulation of transcription and expression, the regulation of protein content and protein modification, and the like. Not only will this provide greater insight into how a cell operates, but it also allows for the determination of when a cell is aberrant or diseased. In addition, one can determine the effect of changes in the environment of the cell on the cellular function, as evidenced by changes in protein profiles, modification of proteins and transport of proteins.

Various approaches have been used to study protein-protein interactions, particularly using yeast as a host. While this can provide information concerning whether two proteins will interact, it gives no information about what happens in a native cell. The use of yeast as a host may also provide information about compounds that interfere with the interaction, but in an environment substantially different from the mammalian natural environment where the interaction may occur.

Other techniques have involved tagging a protein with a peptide fluorescer, e.g., green fluorescent protein, where degradation of the fusion protein can be followed by the loss of the fluorescence. This has many disadvantages in requiring a very large tag that may interfere with the folding of the native protein, its binding to other proteins, its susceptibility to degradation and its overall regulatory activity.

In studying the effect of drugs, both as to efficacy and differences in individual responses, it would be helpful to understand the differences in the individual hosts that result in the different responses. In understanding diseased states, it would be advantageous to be able to compare the changes in protein activity as a result of the cellular diseased state. By providing the capability to monitor changes in one or more proteins, therapeutic, diagnostic and scientific information can be developed.

### BRIEF DESCRIPTION OF RELEVANT LITERATURE

U.S. Pat. No. 6,037,133 describes the use of green fluorescent protein fusion with IκB for measuring IκB degradation. See also, Li, et al., J. Biol. Chem. 1999, 274:21244-50. Douglas, et al., Proc. Natl. Acad. Sci. USA 1984, 81:3983-7 describes the fusion protein of ATP-2 and lacZ.

### SUMMARY OF THE INVENTION

Methods, compositions, kits and genetic constructs are provided for intracellularly monitoring a β-galactosidase small fragment containing fusion protein as surrogate of a protein(s) of interest as to its interactions, status and activity, particularly as to inhibition of expression by double stranded RNA. The compositions comprise a fusion protein comprising a β-galactosidase enzyme donor oligopeptide fused to a surrogate protein that may include the protein(s) of interest in whole or part. In the presence of the β-galactosidase enzyme acceptor, active β-galactosidase can be determined as a measure of the activity, expression level, intracellular location and/or amount of the protein of interest. Double stranded RNA can be formed by transcription from an integrated gene or by addition and transfer across the cellular membrane. The measurement may be intracellular by having a β-galactosidase enzyme acceptor expressed in the cell with substrate present or a lysate may be used. Degradation, binding events, inhibition of expression by double stranded RNA, localization and modification of the protein of interest may be determined by the assay.

### BRIEF DESCRIPTION OF THE FIGURES

FIG. 1 is the enzyme donor amino acid sequence and nucleic acid sequence;

FIG. 2 is a graph of the ED activity in cells unfused and fused;

FIG. 3 is a graph of TNF induced IκB-ED degradation in HeLa Cells;

FIG. 4 is a graph of IL-1 induced IκB-ED degradation in HeLa Cells; and

FIG. 5 is a graph of carbachol induced IκB-ED degradation in SK-N-SH cells.

### DESCRIPTION OF THE SPECIFIC EMBODIMENTS

Systems are provided employing surrogate fusion proteins for determining cellular events, where the surrogate protein serves as a measure of the protein of interest. The system employs various methods and compositions for determining a cellular event, such as the status of a protein(s) of interest. Genetic expression constructs are provided for introducing the genetic construct into a target cell for expression of the fusion protein. The method relies upon the use of a β-galactosidase small fragment, referred to as the enzyme donor (ED), as part of a fusion protein in conjunction with the β-galactosidase large fragment, referred to as the enzyme acceptor (EA), where the complexing of the ED and the EA provide for an active β-galactosidase enzyme. The β-galactosidase activity in the sample acts as a surrogate for the cellular event in the cell as reflected by the activity of the ED in complexing with the EA and forming an active β-galactosidase. Events that result in (1) the expression of the fusion protein or (2) modification of the fusion protein with a change in activity of the ED in complexing or when complexed with the EA, can be measured as an indication of changes in the cell.

The method comprises after performing whatever changes, if any, in environment are to be evaluated, contacting the fusion protein with a β-galactosidase enzyme acceptor in the presence of a detectable substrate, where the β-galactosidase activity is measured. The amount of enzyme product produced is related to the activity of the ED in binding to the EA. The enzyme activity will be influenced by degradation of the fusion protein, binding of the fusion protein to a compound complexing with the protein of interest, modification of the fusion protein, transport of the fusion protein (translocation), and the like. One can also measure the rate of expression, transcription and translation, resulting from a promoter, by having a protease stable fusion protein and the expression level of a protein, as a result of the rate of formation and degradation of the fusion protein. The status of the fusion protein may be related to various cellular pathways, where the protein surrogate being measured is associated with a pathway. In this way one can measure the activation or inhibition of a pathway, where such change in the status of the pathway changes the activity of the fusion protein.

The changes in the activity of the ED can be a result of any of a number of processes that result in a change in the activity of the ED in forming an active complex with EA. One such process is the degradation of the fusion protein, for example by polyubiquitination followed by proteasome degradation, protease degradation, denaturation, or any other degradative process. Alternatively, activity can be modified as a result of complex formation between the protein of interest as represented by the fusion protein and another protein. Activity can also be modified due to modification of the fusion protein, where the modification may result in complexing with another protein, change in the fusion protein conformation, presence of a substituent that changes the activity of the ED, or the like. Also, transport of the fusion protein to a compartment in the cell can result in a change in the measurable activity of the ED. In addition, where the modification affecting the ED activity is part of a pathway, the change in ED activity can be related to the events in the pathway.

The fusion protein may comprise a protein of interest, a fragment of the protein of interest, a different polypeptide to represent the protein of interest or may be an intermediate for measuring some other protein or other activity. By polypeptide is intended molecules primarily composed of amino acids having an amide bond, so as to include oligopeptides of at least 2 amino acids, proteins, glycoproteins, lipoproteins, proteins that are phosphorylated, acetylated, methylated, etc., or any other modification of the peptide.

The system employed by the subject invention comprises: (1) preparing the fusion protein gene and expression construct; (2) introducing the expression construct comprising the fusion protein into a selected cell host; (3) optionally, also introducing an expression construct encoding EA; (4) incubating the transformed cell host under conditions that permit expression and cell viability; (5) (i) adding a β-galactosidase intracellular substrate or (ii) lysing the cell host and adding a β-galactosidase substrate; and (6) measuring the turnover rate of production of β-galactosidase product as a measure of a cellular event, usually as an indication of the status of the protein of interest.

The fusion protein gene expression construct may be used initially to determine whether the gene to be inserted results in a fusion protein that is biologically active to serve as a surrogate for the natural protein. A special host may be used for this determination, as will be discussed below. Once it has been shown that the fusion protein can serve as a surrogate, the construct may then be used in analyzing the status of the protein under conditions of interest.

The first component of the subject invention is the fusion protein and its expression construct. The ED may be at either the C-terminus, the N-terminus or internal to the fusion protein. The particular site of the ED in the fusion protein will depend upon the ability to include the ED in the coding sequence without significant reduction in the natural activity of the protein of interest and without destroying the ability of the ED to complex with the EA to form an active enzyme. Thus, depending upon how much is known about the protein of interest, its structure, site(s) of binding to other entities, the folding pattern, as to loops, β-sheets and α-helices, the manner in which the ED activity will be modulated, e.g. degradation, steric interference of binding with EA by another entity, modification resulting in changes in conformation or charge, etc., the ED will be situated to provide the optimized response. For degradation, it will frequently not matter at what site the ED is situated, this is also likely to be true in many cases for steric interference, so long as the protein of interest retains its natural conformation and susceptibility to degradation. However, for localized modification, such as phosphorylation or dephosphorylation, proteolytic cleavage for maturation, etc., usually it will be desirable to have the ED in proximity to the modified site. By knowing the structure of the protein, one can select loops, α-helices, β-sheets, sites of binding or the like to determine the site for insertion of the ED.

The ED may be inserted into the coding region in a variety of ways. For a cDNA gene, one may select a suitable restriction site for insertion of the sequence, where by using overhangs at the restriction site, the orientation is provided in the correct direction. Alternatively, one may use constructs that have homologous sequences with the target gene and allow for homologous recombination, where the homologous sequences that are adjacent in the target gene are separated by the ED in the construct. By using a plasmid in yeast having the cDNA gene, with or without an appropriate transcriptional and translational regulatory region, one may readily insert the ED construct into the cDNA gene at an appropriate site. Alternatively, one may insert the ED coding region with the appropriate splice sites in an intron or in an exon of the gene encoding the protein of interest. In this way, one can select for a site of introduction at any position in the protein. In some instances, it will be useful to make a number of constructs, where the ED is introduced into an intron and test the resulting proteins for ED activity and retention of function of the protein. Various other conventional ways for inserting encoding sequences into a gene can be employed. For expression constructs and decriptions of other conventional manipulative processes, see, e.g., Sambrook, Fritsch & Maniatis, “Molecular Cloning: A Laboratory Manual,” Second Edition (1989) Cold Spring Harbor Laboratory Press, Cold Spring Harbor, N.Y. (herein “Sambrook et al., 1989”); “DNA Cloning: A Practical Approach,” Volumes I and II (D. N. Glover ed. 1985); “Oligonucleotide Synthesis” (M. J. Gait ed. 1984); “Nucleic Acid Hybridization” [B. D. Hames & S. J. Higgins eds. (1985)]; “Transcription And Translation” [B. D. Hames & S. J. Higgins, eds. (1984)]; “Animal Cell Culture” [R. I. Freshney, ed. (1986)]; “Immobilized Cells And Enzymes” [IRL Press, (1986)]; B. Perbal, “A Practical Guide To Molecular Cloning” (1984).

The gene encoding the fusion protein will be part of an expression construct. The gene is positioned to be under transcriptional and translational regulatory regions functional in the cellular host. The regulatory region may include an enhancer, which may provide such advantages as limiting the type of cell in which the fusion protein is expressed, requiring specific conditions for expression, naturally being expressed with the protein of interest, and the like. In many instances, the regulatory regions may be the native regulatory regions of the gene encoding the protein of interest, where the fusion protein may replace the native gene, particularly where the fusion protein is functional as the native protein, may be in addition to the native protein, either integrated in the host cell genome or non-integrated, e.g. on an extrachromosomal element.

In those cells in which the native protein is present and expressed, the fusion protein will be competing with the native protein for transcription factors for expression. The site of the gene in an extrachromosomal element or in the chromosome may vary as to transcription level. Therefore, in many instances, the transcriptional initiation region will be selected to be operative in the cellular host, but may be from a virus or other source that will not significantly compete with the native transcriptional regulatory regions or may be associated with a different gene from the gene for the protein of interest, which gene will not interfere significantly with the transcription of the fusion protein. However, where one is interested in the transcription of the gene of interest, that is, proteins involved in controlling the induction and transcription of the protein of interest, it will usually be desirable to use the native transcriptional regulatory region.

It should be understood that the site of integration of the expression construct will affect the efficiency of transcription and, therefore, expression of the fusion protein. One may optimize the efficiency of expression by selecting for cells having a high rate of transcription, one can modify the expression construct by having the expression construct joined to a gene that can be amplified and coamplifies the expression construct, e.g. DHFR in the presence of methotrexate, or one may use homologous recombination to ensure that the site of integration provides for efficient transcription. By inserting an insertion element, such as Cre-Lox at a site of efficient transcription, one can direct the expression construct to the same site. In any event, one will usually compare the β-galactosidase activity from cells in a predetermined environment to cells in the environment being evaluated.

There are a large number of commercially available transcriptional regulatory regions that may be used and the particular selection will generally not be crucial to the success of the subject invention. Also, the manner in which the fusion gene construct is introduced into the host cell will vary with the purpose for which the fusion gene is being used. The introduction of the construct may be performed in vitro or in vivo and will include situations where cells transformed in culture are then introduced into the mammalian host. The transcriptional regulatory region may be constitutive or inducible. In the former case, one can have a steady state concentration of the fusion protein in the host, while in the latter case one can provide going from the substantially total absence (there is the possibility of leakage) to an increasing amount of the fusion protein until a steady state is reached. With inducible transcription, one can cycle the cell from a state where the fusion protein is absent to a state where the steady state concentration of the fusion protein is present.

Vectors for introduction of the construct include an attenuated or defective DNA virus, such as but not limited to, herpes simplex virus (HSV), papillomavirus, Epstein Barr virus (EBV), adenovirus, adeno-associated virus (AAV), and the like. Defective viruses, appropriately packaged, which entirely or almost entirely lack viral genes, are preferred. Defective virus is not infective after introduction into a cell. Use of defective viral vectors, particularly tropic for particular cell types, allows for administration to cells in a specific, localized area, without concern that the vector can infect other cells. Thus, a particular locus can be specifically targeted with the vector. Specific viral vectors include: a defective herpes virus 1 (HSV1) vector (Kaplitt et al., 1991, Molec. Cell. Neurosci. 2:320-330); an attenuated adenovirus vector, such as the vector described by Stratford-Perricaudet et al. (1992, J. Clin. Invest. 90:626-630 a defective adeno-associted virus vector (Samulski et al., 1987, J. Virol. 61:3096-3101; Samulski et al., 1989, J. Virol. 63:3822-3828).

The vector may be introduced in vitro and in vivo by lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection. (Felgner, et. al., 1987, Proc. Natl. Acad. Sci. U.S.A. 84:7413-7417; see Mackey, et al., 1988, Proc. Natl. Acad. Sci. U.S.A. 85:8027-8031)). The use of cationic lipids may promote encapsulation of negatively charged nucleic acids, and also promote fusion with negatively charged cell membranes (Felgner and Ringold, 1989, Science 337:387-388). Lipofection into the nervous system in vivo has recently been achieved (Holt, et. al., 1990, Neuron 4:203-214). The use of lipofection to introduce exogenous genes into the nervous system in vivo has certain practical advantages. Lipids may be chemically coupled to other molecules for the purpose of targeting (see Mackey, et. al., 1988, supra). Targeted peptides or non-peptide molecules can be coupled to liposomes chemically.

It is also possible to introduce the vector in vitro and in vivo as a naked DNA plasmid, using calcium phosphate precipitation or other known agent. Alternatively, the vector containing the gene encoding the fusion protein can be introduced via a DNA vector transporter (see, e.g., Wu et al., 1992, J. Biol. Chem. 267:963-967; Wu and Wu, 1988, J. Biol. Chem. 263:14621-14624; Hartmut et al., Canadian Patent Application No. 2,012,311, filed Mar. 15, 1990).

Vectors are introduced into the desired host cells in vitro by methods known in the art, e.g., transfection, electroporation, microinjection, transduction, cell fusion, DEAE dextran, calcium phosphate precipitation, lipofection (lysosome fusion), use of a gene gun, using a viral vector, with a DNA vector transporter, and the like.

Advantages associated with in vivo introduction of the fusion protein expression construct are that one has the expression of the fusion protein in a natural setting where the factors normally associated with the status of the cell are present. For example, if one were interested in knowing how a drug acted on a cell type in relation to the protein of interest, by testing the drug in vivo, one is able to determine the response of the protein of interest under natural conditions.

Expression vectors containing the fusion protein gene inserts can be identified by four general approaches: (a) PCR amplification of the desired plasmid DNA or specific mRNA, (b) nucleic acid hybridization, (c) presence or absence of “marker” gene functions, and (d) expression of inserted sequences. In the first approach, the nucleic acids can be amplified by PCR with incorporation of radionucleotides or stained with ethidium bromide to provide for detection of the amplified product. In the second approach, the presence of the fusion protein gene inserted in an expression vector can be detected by nucleic acid hybridization using probes comprising sequences that are homologous to the fusion protein gene. In the third approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of foreign genes in the vector. In the fourth approach, recombinant expression vectors can be identified by assaying for the activity of the fusion protein gene product expressed by the recombinant expression vector.

One may use promoters that are active for a short time, such as viral promoters for early genes, for example, the human cytomegalovirus (CMV) immediate early promoter. Other viral promoters include but are not limited to strong promoters, such as cytomegaloviral promoters (CMV), SR.alpha. (Takebe et al., Mole. Cell. Biol. 8:466 (1988)), SV40 promoters, respiratory syncytial viral promoters (RSV), thymine kinase (TK), beta.-globin, etc. Alternatively, an inducible promoter can be used.

A large number of promoters have found use in various situations, for various purposes and for various hosts. Many promoters are commercially available today. Expression of the fusion protein may be controlled by any promoter/enhancer element known in the art, but these regulatory elements must be functional in the host or host cell selected for expression. Promoters which may be used to control fusion gene expression include, but are not limited to, the SV40 early promoter region (Benoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42); and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58), alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94), myelin basic protein gene control region which is active in oligodendrocyte cells in the brain (Readhead et al., 1987; Cell 48:703-712), myosin light chain-2 gene control region which is active in skeletal muscle (Sani, 1985, Nature 314:283-286), prostate specific antigen control region, which is active in prostate cells (U.S. Pat. Nos. 6,197,293 and 6,136,792), and gonadotropic releasing hormone gene control region which is active in the hypothalamus (Mason et al., 1986, Science 234:1372-1378). Alternatively, expression of the fusion protein gene can be under control of an inducible promoter, such as metallothionein promoter, which is induced by exposure to heavy metals. For control of the gene transfected into certain brain cells, a glucocorticoid inducible promoter can be used, since glucocorticoids can cross the blood-brain barrier. Alternatively, an estrogen inducible promoter, which would be active in the hypothalamus and other areas responsive to estrogen, can be used. The present invention contemplates the use of any promoter inducible by a pharmacologic agent that can cross the membrane and for neuronal cells, the blood-brain barrier and influence transcription.

Vectors containing DNA encoding the following proteins, for example, have been deposited with the American Type Culture Collection (ATCC) of Rockville, Md.: Factor VIII (pSP64-VIII, ATCC No. 39812); a Factor VIII analog, “LA”, lacking 581 amino acids (pDGR-2, ATCC No. 53100); t-PA and analogs thereof (see co-pending U.S. application Ser. No. 882,051); VWF (pMT2-VWF, ATCC No. 67122); EPO (pRK1-4, ATCC No. 39940; pdBPVMMTneo 342-12 (BPV-type vector) ATCC No. 37224); and GM-CSF (pCSF-1, ATCC No. 39754).

The vector will include the fusion gene under the transcriptional and translational control of a promoter, usually a promoter/enhancer region, optionally a replication initiation region to be replication competent, a marker for selection, as described above, and may include additional features, such as restriction sites, PCR initiation sites, an expression construct providing constitutive or inducible expression of EA, or the like. As described above, there are numerous vectors available providing for numerous different approaches for the expression of the fusion protein in a host.

The host cells will be selected to provide the necessary transcription factors for expression of the fusion protein and the other components for the purposes of the determination. The host cells will also be selected toward providing an environment resembling the environment being simulated. In many cases primary cells may be employed, both those maintained in culture and obtained directly from a patient. However, in many other cases, established cell lines will be used, since the cell lines can provide the desired environment and allow for direct comparisons between studies, which comparisons may not be available where using primary cell lines from patients.

Established cell lines, including transformed cell lines, are suitable as hosts. Normal diploid cells, cell strains derived from in vitro culture of primary tissue, as well as primary explants (including relatively undifferentiated cells such as hematopoietic stem cells) are also suitable. Embryonic cells may find use, as well as stem cells, e.g. hematopoietic stem cells, neuronal stem cells, muscle stem cells, etc. Candidate cells need not be genotypically deficient in a selection gene so long as the selection gene is dominantly acting. The host cells preferably will be established mammalian cell lines. For stable integration of vector DNA into chromosomal DNA, and for subsequent amplification of the integrated vector DNA, both by conventional methods, CHO (Chinese Hamster Ovary) cells are convenient. Alternatively, vector DNA may include all or part of the bovine papilloma virus genome (Lusky et al., 1984, Cell 36:391-401) and be carried in cell lines such as C127 mouse cells as a stable episomal element. Other usable mammalian cell lines include HeLa, COS-1 monkey cells, melanoma cell lines such as Bowes cells, mouse L-929 cells, mouse mammary tumor cells, 3T3 lines derived from Swiss, Balb-c or NIH mice, BHK or HAK hamster cell lines and the like.

Cell lines may be modified by knocking out specific genes, introducing specific genes, e.g. the EA coding gene, enhancing or diminishing the expression of a protein or the like. The modification may be transient, as in the case of introduction of antisense DNA or dsRNA, including RNAi, such as siRNA, or may be permanent, by deleting a gene, introducing a gene encoding the antisense mRNA of the target protein, adding a dominant recessive gene, or the like. These procedures are well established as evidenced by the scientific and patent literature. See, for example, for antisense: Zhang, et al., 2002 J Gene Med 4. 183-94; Shi, et al., 2001 Cancer Biother Radiopharm 16,421-9; Allen and Renzi 20021 Antisense Nucleic Acid Drug Dev 11, 289-300; WO 00/61602; WO99/61462; and WO92/00990; for dsRNA, Heitmeier, et al. 1999 J Biol Chem 274, 12531-6, US2002/0114784 AND WO 01/77350; and a special case of dsRNA, namely iRNA: Agami, 2002 Curr Opin Chem Biol 6, 829-34; Minski, et al., J Biol Chem 277, 49453-8; Malhotra, et al., 2002 Mol Microbiol 45, 1245-54; Sui, et al., 2002 PNAS USA 99, 5515-20; and Yang, et al., 2000 Curr Biol 10, 1191-2000. Methods for introducing the RNA transiently are well known as exemplified by the references cited above. For permanent integration, the methods described in the above references and the description in the Experimental section can be employed.

Research animals may be employed of various strains, where the strains are a result of naturally occurring mutations and breeding or using genetic modifications of embyronic or other cells with a resulting genetically modified host, which may be vertebrate, e.g. mammalian, fish, insect, or the like, or non-vertebrate, e.g. nematode, etc. Knock-out mice are extensively described in the literature. One may use the intact host, tissue from the intact host or cells from the intact host for the purposes of this invention. Illustrative of the development of knockout and knockin mice are Nozawa, et al., Transplantation 2001, 72:147-55; Ferreira, et al., Blood 2001 98:525-32; Kotani, et al., Biochem. J. 2001, 357:827-34; Zhou, et al., Int. J. Radiat. Biol. 2001, 77:763-72; and Chang, et al., Mol. Cell. Endocrinol. 2001, 180:39-46, and references cited therein, to provide only a few of the large number of publications concerning genetically modified mice. In addition one may use hybridomas, where a first cell having the desired gene(s) is fused with an immortalized cell under conditions where the chromosomes from the first cell are stably maintained. The gene(s) could be transcription factors, proteins of interest, e.g. human proteins in a non-human host cell, or provide for enhanced expression of a protein.

The ED is extensively described in the patent literature. U.S. Pat. Nos. 4,378,428; 4,708,929; 5,037,735; 5,106,950; 5,362,625; 5,464,747; 5,604,091; 5,643,734; and PCT application nos. WO96/19732; and WO98/06648 describe assays using complementation of enzyme fragments. The ED will generally be of at least about 35 amino acids, usually at least about 37 amino acids, frequently at least about 40 amino acids, and usually not exceed 100 amino acids, more usually not exceed 75 amino acids. The upper limit is defined by the effect of the size of the ED on the performance and purpose of the determination, the effect on the complementation with the EA, the inconvenience of a larger construct, and the like. The minimum size that can be used must provide a signal that is modulated by the cellular events and that can be determined with reasonable sensitivity. The examples in the Experimental section will provide guidelines as to how to optimize the size of the ED and position the ED in the fusion protein to obtain the desired sensitivity for the assay.

The status of all intracellular proteins can be determined in accordance with this invention to the extent that the fusion protein can serve as a surrogate for a protein of interest, since all proteins will be subject to some modification, e.g. degradation. However, the degradative process is not the only modification, since any modification that changes the ED activity will be subject to detection. These modifications include aggregation, complex formation with one or more proteins, chemical modification, such as the removal or addition of groups, such as acetyl, phosphate, methyl, sulfate, fatty acid esters, alkoxylation, etc. Instead of the chemical modification of the fusion protein, translocation of the fusion protein can result in a change in activity. For the most part, the proteins of interest will be associated with a health function, such as the effect of an infectious disease, genetic defect, mutation, response to a drug, neoplasia, inflammatory response, etc. Thus, the change in the activity of the ED of the fusion protein will be relevant to a physiological function, usually associated with the diagnosis and treatment of mammalian hosts, although there may be other purposes, such as investigation of pathways.

Degradation can be readily distinguished from other modifications by using additional assays. Knowing the activity of the fusion protein with the EA, one can isolate the fusion protein using antibodies or other binding compounds for sequestering the fusion protein and determining the number of fusion proteins. The difference between the activity from the total fusion protein present in the lysate and the observed activity will be an indication of interactions other than degradation of the fusion protein. Intracellularly, one would have to know the amount of the fusion protein during the cellular cycle, so that the signal that is observed can be related to events other than degradation. The total intracellular amount can be determined using a lysate as described above and the signal observed with different amounts of the fusion protein in the absence or presence of modifications graphed to be used for comparison of results with assays.

Also secreted proteins can be determined while they are intracellular. Prior to being transported from the Golgi to the surface membrane, a number of steps must occur and one can determine the number of such molecules in the cell and whether they are complexed with other proteins, e.g. docking protein.

The efficiency of transcription can also be determined by using a fusion protein that is stable, that is, is not subject to significant modification during the period of the assay. By using a stable protein, such as a prion, β-amyloid, synthetic polypeptides, such as using collagen, keratin or elastin motifs, or providing for secretion into a non-proteolytic environment, one can determine the rate of expression from a regulatory region of interest. By using homologous recombination, one can insert the fusion protein to be under the regulatory control of the regulatory region of interest, including promoters, enhancers, etc. Alternatively, one may introduce a construct with the appropriate regulatory region, where the native and constructed expression systems would both be active, while the fusion protein would indicate the effectiveness of the expression system. In this instance, one would usually be interested in the effect of a change, e.g. environment, genome, etc., on the transcriptional activity of the regulatory region. One could then evaluate the effect of an agent on the transduction of a signal as a result of a binding event at the cell surface, the effect of an intracellular inhibitor, or the effect of a second pathway that involves a first pathway. Desirably, the fusion protein would replace one of the copies of the natural gene, so as to have the same environment for transcription.

Of the protein categories of interest, transcription factors, inhibitors, regulatory factors, enzymes, membrane proteins, structural proteins, and proteins complexing with any of these proteins, are of interest. Specific proteins include enzymes, such as the hydrolases exemplified by amide cleaving peptidases, such as caspases, thrombin, plasminogen, tissue plasminogen activator, cathepsins, dipeptidyl peptidases, prostate specific antigen, elastase, collagenase, exopeptidases, endopeptidases, aminopeptidase, metalloproteinases, including both the serine/threonine proteases and the tyrosine proteases; hydrolases such as acetylcholinesterase, saccharidases, lipases, acylases, ATP cyclohydrolase, cerebrosidases, ATPase, sphingomyelinases, phosphatases, phosphodiesterases, nucleases, both endo- and exonucleases; oxidoreductases, such as the cytochrome proteins, the dehydrogenases, such as NAD dependent dehydrogenases, xanthine dehyrogenase, dihydroorotate dehydrogenase, aldehyde and alcohol dehydrogenase, aromatase; the reductases, such as aldose reductase, HMG-CoA reductase, trypanothione reductase, etc., and other oxidoreductases, such as peroxidases, such as myeloperoxidase, glutathione peroxidase, etc., oxidases, such as monoamine oxidase, myeloperoxidases, and other enzymes within the class, such as NO synthase, thioredoxin reductase, dopamine β-hydroxylase, superoxide dismutase, nox-1 oxygenase, etc.; and other enzymes of other classes, such as the transaminase, GABA transaminase, the synthases, β-ketoacyl carrier protein synthase, thymidylate synthase, synthatases, such as the amino acid tRNA synthatase, transferases, such as enol-pyruvyl transferase, glycinamide ribonucleotide transformylase, COX-1 and -2, adenosine deaminase.

Kinases are of great significance, such as tyrosine kinases, the MAP kinases, the cyclin dependent kinases, GTP kinases, ser/thr kinases, Chk1 and 2, etc.

Also of interest are enzyme inhibitors, such as α₁-antitrypsin, antithrombin, cyclophilin inhibitors, proteasome inhibitors, etc.

Other proteins of interest are the oncogenes, such as Src, Ras, Neu, Erb, Fos, Kit, Jun, Myc, Myb, Abl, Bcl, etc. Cytokines, such as the interferons, α-γ, interleukins, 1-19, and integrins, adhesins, TNF, receptors, hormones, colony stimulating factors, growth factors, such as epidermal growth factor, fibroblast growth factor, etc., bone morphogenetic proteins, developmental proteins, such as the Hox proteins, or other proteins binding to or regulating proteins binding to homeoboxes, e.g. the hedgehog proteins, basement membrane proteins, heat shock proteins, proteins containing Krupple and Kringle structures chaperonins, calcium associated proteins, e.g. calmodulin, calcineurin, etc., membrane channels, transporter proteins, etc.

Also of interest are the proteins associated with proliferation, such as the cyclins, cyclin dependent kinases, p53, RB, etc.

Neuronal proteins, such as β-amyloid, TNF, prion, APP, transporters, e.g. dopamine transporter, receptors, such as NMDA receptors, AMDA receptors, dopamine receptors, channels, etc.

Another class of proteins is the transcription factors and their inhibitors or regulatory proteins, such as Adr Ace, Amt, AP, Atf, Att, Baf, Bm, Btf, C Ebp, C Jun, C Ets, CREB, CF, Chop, DP, E2F, Elk, Gata, Hnf, Iii A-H, Irf, NY Y, Otf, NFκB, NF-AT, Oct-1, Pea, Pit, PU, S, SP, Stat, Tef, TFIII, TFIIII, Ubf and Usf, while the inhibitors include Erk, IκB, LIF, Smad, RANTES, Tdg, etc., as well as other proteins associated with pathways that induce transcription factor synthesis, activation or inhibition.

Another class of proteins that are of interest are the surface membrane proteins, where many of such proteins are receptors, such as G protein complex receptors, hormone receptors, interleukin receptors, steroid receptors, transporters, etc. These receptors include insulin receptor, glucose transporter, IL-2, 4, etc. receptor, CRXC4, PPAR, etc. Also, the MHC proteins can be of interest.

In some instances, housekeeping proteins will be of interest, such as the proteins involved in the tricarboxylic acid cycle, the Krebs cycle, glycogenesis, etc.

As indicated previously, the genes of each of these proteins may be manipulated in numerous ways to fuse ED with the protein while maintaining the biological activity of the protein and ED.

Various pathways will be of interest associated with the different proteins. Thus, pathways involving signal transduction as a result of ligand binding to a surface membrane protein receptor, vesicle formation and transport, multistage synthesis of cellular components, proteasomes, peroxisomes, spindle formation, tubulin assemblage, processing of ingested compounds, e.g. toxins, drugs, etc.

The cells comprising the subject constructs may be used to identify proteins associated with a pathway of interest, the effect of a change in environment, such as the presence of a drug or drug candidate, on the production of the protein of interest, changes in the regulation of expression, the effect of inhibiting expression of a protein, the regulation by a receptor of a cellular pathway and to that extent, compounds that affect the transduction of a signal by the receptor, the activation or deactivation of cellular pathways that affect the complex formation or degradation of the fusion protein, expression level of a protein, related to the rates of formation and degradation, etc.

A number of substrates for β-galactosidase are known, where the product is fluorescent. The common substrates are β-D-galactopyranosyl phenols, such as fluorescein, mono- and di-susbtituted, o-nitrophenyl-β-D-galactoside, β-methylumbelliferyl-β-D-galactoside, X-gal, resorufin-β-D-galactoside, commercially available oxetanes, e.g. Galacto-Light Plus® kits (chemiluminescence) and chlorophenol red. The di-β-D-galactopyranosylfluorescein, and chlorophenol red-β-D-galactopyranoside, or analogous substrates, particularly where the product is inhibited from leaking from the cell, may be used as intracellular markers.

During the determination, the cells are maintained in a viable state, where the cells may be dividing or not dividing. The viable state may be referred to growing. The determination may be made with intact cells or a cellular lysate.

The simplest procedure to describe is the use of cells in culture and analysis of the lysate. In this case, the cells are grown in culture. The fusion protein and other constructs, as appropriate, may be present in the cell integrated into the genome or may be added transiently by the various methods for introducing DNA into a cell for functional translation. The cells may be in culture or in vivo. These methods are amply exemplified in the literature, as previously described. By employing a marker with the fusion protein for selection of cells comprising the construct, such as antibiotic resistance, development of a detectable signal, etc., cells in culture comprising the fusion protein can be separated from cells in which the construct is absent. Once the fusion protein is being expressed, the environment of the cells may be modified, if desired. Candidate compounds may be added, ligand for receptors, surface membrane or nuclear, or the two of these may be added in combination, changes in the culture medium may be created, other cells may be added for secretion of factors or binding to the transformed cells, viruses may be added, or the like. Given sufficient time for the environment to take effect and/or taking aliquots of the culture at different time intervals, the cells may be lysed with a lysis cocktail comprising EA and enzyme substrate and the signal from the product read. One can then relate this result to the amount of fusion protein present, particularly by using standards where the lysate is spiked with different amounts of the fusion protein and the amount of active fusion protein determined. One would then have a graph relating signal to amount of active fusion protein in the lysate.

Where the cells are in a viable host, usually the cells or tissue from the host will be harvested and may be lysed, so that the methodology used for the culture will be the same. Selection of cells having the construct can be achieved by having an antibiotic resistance gene as part of the construct, so that cells can be selected using the antibiotic to avoid dilution of the sample by cells lacking the construct.

With intact cells, the cells are maintained in the culture, during which time the fusion protein and EA are expressed intracellularly, either transiently, constitutively or inducibly. Also, the substrate will be maintained, usually in the medium at a concentration where the substrate in the cell is at a concentration that will permit detection of changes in the activity of the fusion protein relevant to the assay. In some instances, one can inject the substrate into the cell using any conventional technique or provide for permeabilization of the cell, followed by washing and curing the membrane, so as to lock the substrate intracellularly. The cells can be analyzed by FACS, cytologically, electrophoretically, fluorimetrically, etc.

The subject methodology may also be used in determining ligands for receptors, where a member of the pathway regulated by the receptor is known. This may be exemplified by the GPCRs, where IκB is a member of the regulatory system in the transduction of the signal from the receptor. By using a cell where the receptor is present in a functional state and the fusion protein simulates a member of the pathway, where the up or down regulation of the member of the pathway is related to binding of the ligand to the receptor, by determining the amount of the fusion protein, one can relate the amount to the activity of the candidate compound as a ligand. For example, with GPCRs, activation of the GPCR by ligand binding results in the degradation of IκB, so that an active ligand will result in the degradation of the fusion protein. The screening may be multiplexed providing protection against an antagonist making an agonist.

For convenience, kits can be provided that may include all or some of the major components of the assays. For example, a kit may include an expression construct, by itself or as part of a vector, e.g. plasmid, virus, usually attenuated, where the expression construct may include a marker, a gene encoding a protein for integration, a replication initiation site, and the like. In addition to the expression construct, the kit may include EA, substrate for β-galactosidase, one or more cell lines or primary cells, a graph of response in relation to the amount of ED present, buffer, etc. In some instances cells may be engineered to provide a desired environment, such as high levels of expression of a protein involved in a pathway of interest, such as surface membrane receptors, GPCRs, nuclear receptors, e.g. steroid receptors, transcription factors, etc. or may have been mutated, so as to have reduced levels of expression affecting the expression of the fusion protein and one is interested in enhancing the level of expression. The system is initially used to determine whether the gene to be inserted results in a fusion protein that is biologically active to serve as a surrogate for the natural protein. The activity of the fusion protein may be determined by using host cells in which the expression of the natural protein does not occur, such as cells in which both copies of the natural protein have been knocked-out, where antisense RNA is added to the host cell that inhibits the natural protein but not the fusion protein, e.g. as to the non-coded 3′-region or includes the 5′-methionine codon, inhibits a transcription factor necessary for the natural protein, where the fusion protein has a different transcriptional regulatory region, if an enzyme, is shown to bind to its natural substrate and catalyze its reaction at a rate reasonably commensurate with the natural enzyme or, if not an enzyme, binds with an appropriate affinity to the proteins the natural protein binds to, etc.

In developing the use of the fusion proteins, systems can be devised that allow for screening of genetic constructs for their application to the desired investigation. The user of the system introduces the gene of interest into the genetic construct provided in the system. By having a multiple cloning site, the gene is manipulated so as to be inserted into the multiple cloning site in the correct orientation and in reading frame with the ED sequence. Usually, there will be a linker of not more than 3 codons, preferably not more than about 2 codons, as a result of the nucleotides present in the multiple cloning site remaining between the ED sequence and the gene of interest. As indicated, the vector that is provided may include the transcrptional and/or translational termination sequences, a polyadenylation sequence, or other sequence that encodes a function, e.g. famesylation, geranylation, etc. Once the fusion protein construct has been completed, the construct may then be introduced into the host cell. The host cell may have a construct expressing the EA or, if not, such a construct may be added for transient expression or for integration into the genome and stable expression. Alternatively, a lysate may be prepared and, as appropriate, the EA added. The substrate that is chosen may be able to permeate the cell membrane, so that the substrate will be present in the cell in a non-rate-limiting amount. Alternatively, as indicated above a lysate may be prepared and the substrate added to the lysate.

After the necessary modifications of the host cells have been accomplished, one may then proceed with the use of the host cells. For example, after performing whatever changes, if any, in environment of the host cells are to be evaluated, one would contact the fusion protein with an EA in the presence of a detectable substrate, where the enzyme activity is measured. The amount of enzyme product produced is related to the activity of the ED in binding to the EA. The enzyme activity will be influenced by degradation of the fusion protein, binding of the fusion protein to a compound complexing with the protein of interest, modification of the fusion protein, transport of the fusion protein, and the like. One can also measure the rate of expression, transcription and translation, resulting from a promoter, by having a protease stable fusion protein and the expression level of a protein, as a result of the rate of formation and degradation of the fusion protein.

The changes in the activity of the ED can be a result of the degradation of the fusion protein, by ubiquitination followed by degradation, protease degradation, denaturation, or other process. Alternatively, activity can be modified as a result of complex formation between the protein of interest and another protein. Activity can also be modified due to modification of the fusion protein, where the modification may result in complexing with another protein, change in the fusion protein conformation, presence of a substituent that changes the activity of the ED, or the like. Also, transport of the fusion protein to a compartment in the cell can result in a change in the measurable activity of the ED. In addition, where the modification affecting the ED activity is part of a pathway, the change in ED activity can be related to the events in the pathway. The fusion protein may comprise a protein of interest, a fragment of the protein of interest, a different polypeptide to represent the protein of interest or may be an intermediate for measuring some other protein or other activity.

Protein transport or translocation in the cell from the nucleus to another organelle or site, e.g. cytosol, cell membrane, proteasome, mitochondria, lysosome, Golgi, etc., can be of great importance to the biological properties of the protein and the cellular pathways of the cell. For protein transport, one can use leader sequences at the N terminus of the fusion protein from proteins that are known to be translocated to particular sites. One may also use coding sequences that result in modification of the fusion protein for binding the fusion protein to the cell membrane, such as sequences resulting in prenylation, myristoylation, farnesylation, etc. By providing for EA and substrate in the cell, depending upon the site of the fusion protein, one may be able to detect the presence of the fusion protein at the particular site.

The steps employed by the subject invention comprise: (1) preparing the fusion protein gene and expression construct by insertion of the gene of interest into the multiple cloning site of the genetic construct provided as part of the system; (2) introducing the expression construct comprising the fusion protein into a selected cell host, provided by the system or selected by the user; (3) optionally, also introducing an expression construct encoding EA, if not previously present as part of the host cells provided with the system; (4) incubating the transformed cell host under conditions that permit expression and cell viability; (5) (i) adding an intracellular substrate or (ii) lysing the cell host and adding EA and a substrate; and (6) measuring the turnover rate of production of product as a measure of a cellular event, usually as an indication of the status of the protein of interest. When providing for expression of EA, one will generally use a highly active promoter to ensure that there is a sufficient amount of the EA present in the cell to complex substantially all of the ED present, thus the EA promoter should be at least about twice as active as the ED promoter. When adding EA to the lysate, the same consideration is present, so that usually a large excess of EA to ED will be added, usually at least about two-fold excess, frequently at least about five-fold excess, and the excess may be 20-fold or greater.

Using the various components described above for use in this invention, the system can be employed to determine whether the host and expression construct are compatible, whether transient expression, extended expression, or permanent expression should be employed, whether primary, immortal or cells of intermediate nature should be employed, and the response to known agents. In this way, one can optimize the particular system to provide for increased sensitivity to particular agents.

The system can be used with a data accumulation and storage capability, where the data derived from the system is collected, analyzed and compared to other determinations. In this way, data can be accumulated of the effect of various agents on the activity of the ED, so that one can measure how the genetically modified cells respond to the addition of individual or combinations of candidate drugs, variations in rate of change, affect on different pathways and the like. By having a database of known responses to compounds that have established effects, new candidates can be compared to such results for evaluation of the their anticipated physiological effects. Not only can one study the effect of such candidates on targets, but also the side effects resulting from the presence of such targets in the media.

As indicated, the subject method can be used in a variety of situation to great effect, since the ED is small enough to allow for functioning of the protein of interest as a fusion protein with ED, while allowing for ED to complex with EA to provide a functional β-galactosidase.

The following examples are offered by way of illustration and not by way of limitation.

### Experimental

### Generation of IκB-ED Fusion Protein

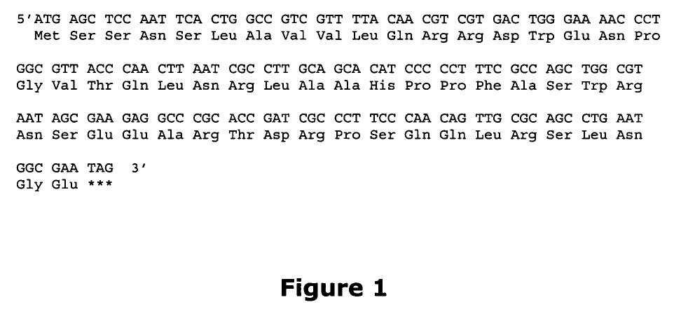

The cDNAs encoding IκB and ED (FIG. 1) were amplified with Pfu DNA polymerase (Stratagene, CA). Both IκBα and IκB M were amplified using forward primer: 5′-CCGAAGCTTATGTTCCAGGCGGCCGAG-3′ and reverse primer: 5′-ATAGGATCCTAACGTCAGACGCTGGCC-3′. These primers incorporated a Hind III at the 5′ end and a Bam HI at the 3′ end of the PCR products. Also, the stop codon of the IκB was removed in order to provide an open reading frame with ED. pCMV-IκB and pCMV-IκB M (CLONTECH, CA) was used as PCR template. IκB M contains a serine to alanine mutation at amino acid residue 32 and 36. These two sites are critical to the phosphorylation of IκB, and the mutant results in the resistance of IκB to degradation (Ref . . . ). ED, on the other hand was amplified using forward primer: 5′-ATAGGATCCATGAGCTCCAATTCACTGGCCG-3′ and reverse primer 5′-ATAAGAATGCGGCCGCCTATTCGCCATTCAGGCTGCGC-3′. The forward primer incorporated a Bam HI site to the ED and the reverse primer incorporated a Not I site to the ED as well as a stop codon. The amplification was using the PCR program with denature DNA at 92° C. for 1 min, anneal at 52° C. for 1 min and then elongate at 72° C. for 2 min, followed by 29 cycles repeating in total. The amplified PCR products were ligated at the Bam HI site and the resulting fusion constructs were subcloned into a mammalian expression vector pCMV at the sites of Hind III and Not I resulting in the construct designated pCMV-IκB-ED. pCMV vector originated from pCMV-IκB α (CLONTECH, CA), where the IκB α was substituted by IκB-ED fusion construct. The pCMV-ED construct was obtained by inserting ED PCR product into the Bam HI site and Not I site following standard molecular biology procedure (Maniatis et al;).

### Expression of ED Fusion Proteins in Cell Culture

HeLa cells were kept in culture in DMEM medium (GIBCO, CA) supplemented with 10% fetal bovine serum and 2 mM glutamine (GIBCO, CA). For transient transfection, cells were seeded into 6 well plate one day before experiment. For each well, 3 μl of Fugene 6 (Roche, Ind.) was diluted into 100 μl of serum free medium, and then 1 μg of plasmid DNA was added. The mixture was incubated at room temperature for 15 min before addition into wells dropwise. The plate was then incubated at 37° C. till the assay.

To detect ED activity, 24 hr after transfection, the culture medium was removed, and the cells lysed with 200 μl of cell lysis buffer (0.5% CHAPS, 10 mM potassium phosphate, 10 mM sodium chloride, pH 6.9). Then 30 μl of the cell lysate was transferred into 384-well plate, where 10 μl of EA reagent (0.18 mg/ml EA and 0.5% fetal bovine serum in EA core buffer (100 mM PIPES, 400 mM NaCl, 1 mM EGTA, 0.005% Tween, 150 mM NaOH, 10 mM Mg acetate, 14.6 mM NaN₃, pH 6.9)) was added. After 30 min incubation at room temperature, 15 μl of chemiluminescence substrate (4% of Galacton Star™ and 20% of Emerald II™ (Tropix) in EA core buffer) was added. The signal was read on Lumicount (Packard) or Fluoroskan (Labsystem) with integration time of 1 second per well.

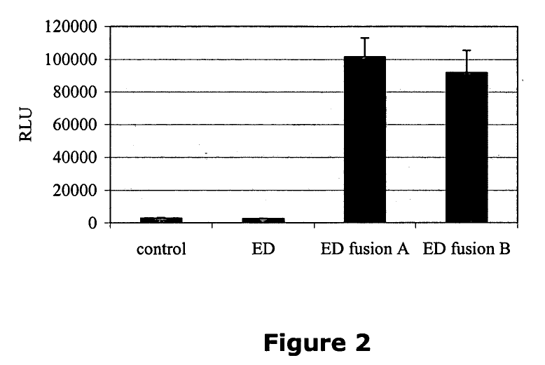

In FIG. 2, three constructs were transfected into HeLa cells, they are pCMV-ED, pCMV-IκB-ED and pCMV-IκB M-ED, respectively. The non-transfected cells were also used as negative control. When ED was expressed as a fusion protein, the ED activity is readily detected, indicating that the fusion protein is relatively stable. However, when the ED is expressed alone, unfused, the ED activity dropped to the basal level, suggesting that the unfused ED is a very unstable peptide, and gets degraded quickly in cells.

### TNFα Induced IκB-ED Degradation in HeLa Cells

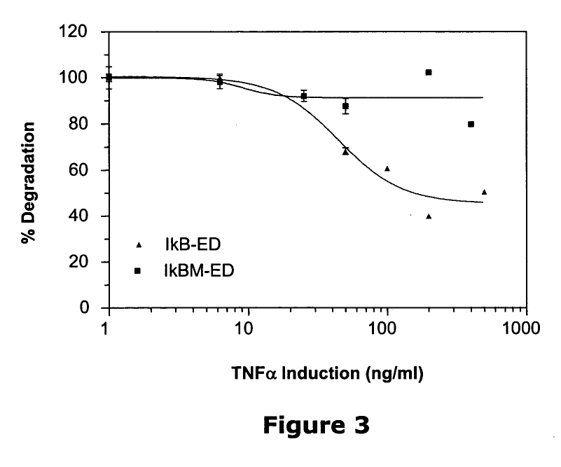

HeLa cells were seeded into 24 well plate 24 hr before transfection. 0.25 μg DNA was transfected into each well using Fugene6 (Roche) following manufacture's protocol. 24 hr after transfection, cells were subjected to treatment of TNFα (Sigma) at various concentrations for 30 min. Then the culture medium was removed, and cells were lysed in 90 μl of cell lysis buffer. 30 μl of cell lysate was transferred into 384 well plate, where 10 μl EA reagent was added. Assay was performed in three replicates. The plate was incubated at room temperature for 30 min before addition of 15 μl chemiluminescence substrate. Plate was read 30 min after substrate addition. The untreated cells were normalized to 100% activity. As shown in FIG. 3, TNFα was able to decrease the ED activity in a dose dependent manner, which indicated the degradation of wild type IκB. Contrastingly, the mutant form did not show a dose dependent decrease of ED activity upon TNFα treatment, as expected. This result demonstrated that ED as a fusion tag did not change the IκB biological function, and was able to monitor IκB degradation in vivo. In addition, the IκB-ED degradation was specifically linked to the upstream component activation. Also confirmed was that the IκB-ED degradation was dependent on IκB phosporylation at 32 and 36 residues, the same way as un-tagged IκB.

### IL-1 Induced IκB-ED Degradation in HeLa Cells

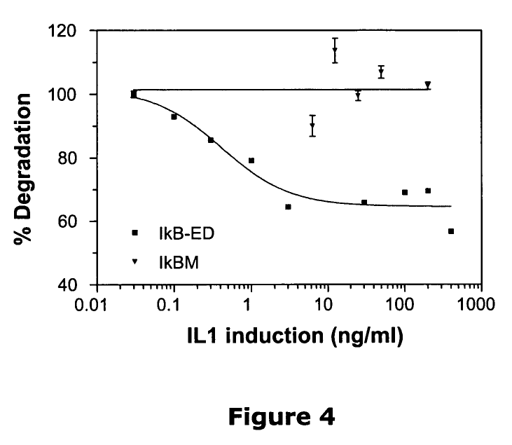

It has been reported that IL-1 activation of cells results in NF-κB pathway activation through the induced degradation of IκB. To confirm that the ED labeled IκB in cells was able to monitor IL-I pathway activation, HeLa cells was transiently transfected with pCMV-IκB-ED or pCMV-IκB M-ED the same way as described above in 24 well plates. Cells were then treated with IL-1 (Sigma) at various concentrations for 30 min then assayed for ED activity. As shown in FIG. 4, IκB-ED activity was decreased upon IL-1 treatment in a dose dependent manner, whereas the mutant form of IκB, IκB M-ED was resistant to IL-1 induced degradation. This result demonstrated that the IκB-ED expressed in HeLa cells was able to be used to monitor endogenous IL-1 receptor activation.

### Gq Coupled GPCR Activation in Neuroblastoma Cell Line SK-N-SH

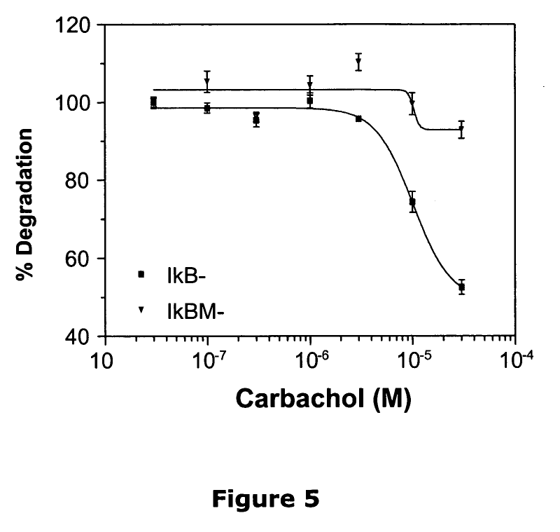

It has been reported that Gq coupled GPCR receptor activation results in NF-κB pathway activation. To demonstrate that the IκB-ED fusion protein can be used as a functional marker to monitor GPCR activation, the neuroblastoma cell line SK-N-SH was used. This cell line was reported to express M3 receptors endogenously. This receptor is Gαq coupled. Carbachol is known as a non-selective agonist to activate M3 receptors. SK-N-SH (ATCC) cells were cultured in MEM medium (Gibco) supplemented with 10% fetal bovine serum and 2 mM glutamine. Cells were seeded into 24 well plates one day before experiment. Then 0.25 μg DNA per well was used to transfect the cells with Fugene6. 24 hr after transfection, cells were treated with carbachol for 20 min at 37° C. Then the cells were lysed and ED activity was assayed as described above. As shown in FIG. 5, carbachol induced M3 activation was indicated by the degradation of IκB-ED, resulting in a decreased RLU reading. The untreated cells were normalized to 100% activity. Upon 30 μM treatment of carbachol, only 50% of the ED activity was retained. The decrease of RLU indicated the induced degradation of IκB-ED fusion protein. The IκB M-ED, on the other hand, did not show the dramatic decrease in activity. More than 92% of ED activity was still retained after treatment with 30 μM carbachol on IκB M-ED expressing cells. This result demonstrated that the ED labeled IκB can also be used to monitor GPCR activation.

The modified cells can be used for a number of purposes. The cells can be used for measuring NF-κB pathway activation, where the cells can be seeded in microtiter plates, treated with a candidate compound, incubated at 37° C. for 30 min and then lysed. After addition of EA and substrate, the generated signal will indicate the effect of the candidate compound on the NF-κB pathway. A signal decrease would indicate that the candidate compound stimulates the pathway. Candidate compounds can be screened for their effect on receptor-ligand interaction, where the receptor-ligand interaction naturally leads to NF-κB pathway activation. The receptor can be co-expressed with the IκB-ED fusion protein in cells or the IκB-ED fusion protein construct can be expressed in cells that express the receptor. The cells are treated with the candidate compounds before addition of the ligand. Inhibition of IκB-ED degradation indicates the inhibition of the receptor activation. Receptors may include those GCPRs, that is, receptors complexed with GTPases, orphan receptors, or any receptor that is coupled to the NF-κB pathway. The protocols can also be used in screening for genes related to the NF-κB pathway. A cDNA expression library can be transfected into cells expressing IκB-ED and any changes in IκB-ED degradation determined. A change in the level of degradation indicates that the gene affects the NF-κB pathway. In this way one can assay for gene function, drug target validation and determining new drug targets. In addition, one may analyze for IKK kinase or ubiquitin pathway activation or inhibition.

By preparing genes for cyclin-ED fusion proteins and transfecting cells with the constructs, one can monitor the cyclin changes as an indicator of cell growth and the effect of candidate compounds on the cyclin, e.g. compounds that control abnormal growth, such as with cancer cells. ED can be fused to p53 and the fusion protein level observed with cell apoptosis, p53 gene modification and p53 accumulation or diminution in the cells.

It is evident from the above results that the subject invention provides a powerful tools for investigating cellular function, affects of agents on cellular function, identification of targets in cells, identification of interactions between cell components, screening of drug candidates, affects of changes in cellular status, such as differentiation, neoplasia, mitosis, meiosis, etc., on the cellular pathways, and the like. The method is straightforward using available components. Fusion proteins are readily prepared and where degradation is involved, the ED can be joined at either end. Where interactions between proteins are of interest, it is feasible to introduce the ED at various sites in the protein without interfering with the biological activity of the fusion protein being measured or the ED. Other applications for the subject invention are also available, in monitoring idiosyncratic responses to drugs, response to treatments, changes in cells, etc.

All publications and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method for analyzing in a cell for the effect on expression of an expression inhibiting nucleic acid, where the nucleic acid interacts with mRNA using an expression construct expressing a fusion protein of the small enzyme donor (ED) fragment of β-galactosidase with a polypeptide, where said expression inhibiting nucleic acid affects the activity of β-galactosidase resulting from said ED forming a functional enzyme with the large enzyme acceptor (EA) fragment of β-galactosidase, said method comprising:
maintaining a cell comprising said expression construct and said expression inhibiting nucleic acid providing said EA to any of said fusion protein produced in said cell to form β-galactosidase, and a β-galactosidase substrate that produces a detectable product; and
determining the activity of said functional enzyme by use of said detectable product,
whereby the activity of said functional enzyme is related to said effect on expression.

2. A method according to claim 1, wherein said effect on expression is the inhibition of expression by a DNA molecule.

3. A method according to claim 1, wherein said nucleic acid is an RNA molecule

4. A method according to claim 3, wherein said RNA molecule is dsRNA

5. A method according to claim 4, wherein said dsRNA is RNAi.

6. A method according to claim 1 wherein said cell is grown in the presence of a candidate compound.

7. A method according to claim 1, wherein said cell is a mammalian cell.

8. A method for analyzing in a cell the effect on expression of an expression inhibiting RNA where the RNA interacts with mRNA using an expression construct expressing a fusion protein of the small enzyme donor (ED) fragment of β-galactosidase with a polypeptide, where said effect on expression affects the activity of said ED in forming a functional enzyme with the large enzyme acceptor (EA) fragment of β-galactosidase, said method comprising:
maintaining a cell comprising said expression construct and said expression inhibiting RNA;
providing said EA to any of said fusion protein produced in said cell to form β-galactosidase, and a β-galactosidase substrate that produces a detectable product; and
determining the activity of said functional enzyme by use of said detectable product,
whereby the activity of said functional enzyme is related to said transcription in said cell.

9. A method according to claim 8, wherein said RNA is double stranded RNA

10. A method according to claim 9, wherein said RNA is RNAi.

11. A method according to claim 8, wherein said expression inhibiting RNA is added to said cell.

12. A method according to claim 8, wherein said expression inhibiting RNA is transcribed in said cell.

13. A method according to claim 8, wherein said substrate produces a fluorescent product.

14. A method according to claim 8, wherein said cell is a cell line.

15. A method according to claim 8 wherein said cell is grown in the presence of a candidate compound.

16. A method according to claim 8, wherein said cell is lysed prior to said determining and said determining is of said lysate.

17. A method according to claim 8 wherein said expression inhibiting RNA inhibits expression of a transcription factor.

18. A system for determining in mammalian cells the effect of an expression inhibiting dsRNA on expression of a first protein where the dsRNA interacts with mRNA, employing a fusion protein comprising a β-galactosidase enzyme donor (“ED”) fused to a second protein, where said first and second proteins are related in that the level of expression of said first protein fusion protein are interrelated, said determining comprising measuring the β-galactosidase activity of said fusion protein in the presence of an enzyme acceptor (“EA”) capable of being complemented by said ED of said fusion protein to form a functionally active β-galactosidase enzyme, said system comprising:
(1) a vector comprising a first transcriptional and translational regulatory region functional in said host cell, (2) an ED sequence encoding said ED joined to a multiple cloning site (“mcs”) under the regulation of said transcriptional and translational regulatory region; the same or different vector as (1) comprising a second transcriptional regulatory region functional in said host cell and a gene encoding said inhibiting dsRNA under the regulation of said transcriptional regulatory region; (3) an enzyme acceptor protein; (4) a gene when inserted in said mcs in reading frame with said ED sequence expresses a biologically active protein and an ED capable of complementing said EA; (5) host cells in which said transcriptional and translational region is functional; and (6) substrate for said β-galactosidase enzyme that upon hydrolysis produces a detectable signal.

19. A system according to claim 18, wherein said first and second transcriptional regulatory regions have the same transcription factors.

20. A system according to claim 19, wherein said first and second transcriptional regulatory regions have different transcription factors.

21. A system according to claim 18, wherein said host cell expresses EA.

22. A kit for use in a method according to claim 1 comprising: an expression construct of the small enzyme donor fragment of β-galactosidase fused to a protein of interest, an expression inhibiting double stranded RNA for said protein of interest, and at least one of an enzyme acceptor fragment of β-galactosidase or a β-galactosidase substrate producing a detectable product.
